(19) United States
(12) Patent Application Publication
(10) Pub. No.: US 2022/0249819 A1
(43) Pub. Date: Aug. 11, 2022

(54) MICRONEEDLES, AND METHODS FOR THE MANUFACTURE THEREOF

(71) Applicant: UNIVERSITY COLLEGE CORK - NATIONAL UNIVERSITY OF IRELAND, CORK, Cork (IE)

(72) Inventors: Waleed FAISAL, Cork (IE); Abina CREAN, Co. Cork (IE)

(21) Appl. No.: 17/617,382

(22) PCT Filed: Jun. 16, 2020
(86) PCT No.: PCT/IE2020/050001

§ 371 Date: Dec. 8, 2021

(30) Foreign Application Priority Data
Jun. 10, 2019 (GB) 1908229.6

(51) Int. Cl. A61M 37/00; A61K 31/4174; A61K 31/496; A61K 9/00; A61P 31/10; A61K 31/4196; A61K 31/415; A61K 31/196; A61K 31/192; A61K 31/519; A61K 31/4422

(52) U.S. Cl. CPC A61M 37/0015; A61K 31/4174; A61K 31/496; A61K 9/0021; A61P 31/10; A61M 2037/0053; A61K 31/415; A61K 31/196; A61K 31/192; A61K 31/519; A61K 31/4422; A61K 31/4196

(57) ABSTRACT

A microneedle with very high drug loading is described, and comprises a base and a penetrating tip, the tip having a dimension ranging from about 50 nm to about 50 μm, wherein at least 80% of the microneedle by volume consists of heat-meltable active pharmaceutical ingredient (v/v). The meltable active pharmaceutical ingredient is characterised by being heat-meltable (i.e. it can be heated to a molten form), is solid at 25° C., and has an ability to form a glassy, amorphous form following melting by heating and cooling with a glass transition temperature greater than 25° C. A method of fabricating a microneedle comprises the steps of providing a microneedle micromold comprising a micromold substrate and one or more holes in the upper surface of the micromold substrate, wherein the interior surface of the hole in the micromold substrate defines an exterior surface of the microneedle, moulding a meltable drug in the microneedle micromold to form a microneedle, and separating the microneedle from the microneedle micromold. Microneedles of the invention may incorporate up to 99% drug or more.

### FIELD OF THE INVENTION

The present invention relates to microneedles and microneedle drug delivery system (DDS). Also contemplated are methods of manufacture of microneedles and microneedle drug delivery system (DDS)

### BACKGROUND TO THE INVENTION

Transdermal or intradermal administration can represent a useful route for drug and vaccine delivery due to the ease of access and avoidance of macromolecular degradation in the gastrointestinal tract. Microneedles have become a safe and relatively pain-free alternative to hypodermic needles for transdermal drug delivery. Traditional materials used in the fabrication of microneedles, metals and synthetic polymers, are associated with various restrictions, however, that compromise their production and performance.

Microneedle (MN) arrays have been fabricated from metals [1], silicon [2], polymers [3] and zeolite [4]. To achieve the drug delivery through the skin many different techniques were applied. MN manufacture and the deposition of the arrays in the skin after applications are the main concerns of the use of silicone and metals. The skin could be firstly pre-treated with solid MN to make microchannels in the skin which facilitate the drug diffusion [5]. This step followed by conventional application of the medication (topical or patch). The two-step application process may lead to practicality issues for patients. Recently-developed microneedle systems employ room temperature processing by coating solid metallic microneedle structures with polymer (a blend of carboxymethylcellulose, Lutrol F-68NF and D-(+)-trehalose dihydrate) containing an influenza vaccine, which deposited in the skin after MN application [6, 7]. While the activity of the incorporated vaccine can be partially preserved during processing, the coating approach to microneedle drug loading provides only a small volume to entrap therapeutic substances compared to bulk loaded structures. Hollow MN is a technique very similar to the traditional injection needles, where the MN array applied to the skin and the drug flow from the reservoir through the hollow by the different in osmotic pressure. The clogging of the needle openings with tissue during insertion and the flow resistance, due to tissue compressed around the MN tips during insertion are the main limitation of this technique. In addition to the high cost of the fabrication procedures, all metal-based microneedle systems have limitations that compromise their function, such as the risk of breaking if improperly applied and the possibility of an inflammatory response or infection if small metal structures remain in the skin.

One current microneedle technology utilizes a dissolvable MN, where the drug is incorporated in a dissolvable biocompatible polymer and start to release in the skin after as the polymer dissolves [8]. In this technique, the moulds are filled with a solution of both the polymer and the drug in a suitable solvent, and the MN array formed after the solvent evaporation. Vacuum or centrifugation is required in a multi-step filling procedure to ensure that the polymer fully occupy the holes in the mould and form reproducible MNs. Bulk-loaded microneedles have been fabricated from biocompatible and dissolvable stabilising materials such as PVP [3], and PLGA [9]. Sugars and sugar derivatives (dextrose [10], maltose [11], galactose [12], (CN103181887, US201412881, KR20160139759, WO2010110397, JP2013111104 and KR101501283) and carboxymethylcellulose [13]) have been also explored. Only relatively low doses of drug can be administered due the requirement for stabilising materials of this dissolvable system, limiting commercialization opportunities. Moreover, microneedles made this way may exhibit poor biocompatibility due to the polymers employed and harmful residues resultant from a fracture of the microneedles in the skin.

A recent approach of using hydro-gel forming polymers or the swelling technique [14], where after the insertion of MN into the skin, the arrays absorb water from interstitial fluid and form hydrogel matrix and the drug starts to diffuse from the drug reservoir. The hydrogel-forming MNs are then removed from skin. The drug dose not only limited to what can be loaded in or on the MN, but more limited to the solubility and diffusion through the hydrogel.

The compounds delivered by MN's to date have typically been of high potency, meaning only a low dose is required to achieve a therapeutic effect [15]. Clearly, the majority of marketed drug substances, including many therapeutic antibodies, are not low dose, high potency molecules. Indeed, many drugs require doses of several hundred milligrams per day in order to achieve therapeutic plasma concentrations in humans. Up till now, such high doses could not be delivered transdermally from a patch of a reasonable size, even for molecules whose physicochemical properties are ideal for passive diffusion across the skin's stratum corneum barrier. Therefore, transdermal delivery has traditionally been limited to fairly lipophilic, low molecular weight, high potency drug substances. Since most drugs do not possess these properties, the transdermal delivery market has not expanded beyond around 20 drugs. Marketed MN-based patches are likely to increase this number of drugs in the coming years. However, this increase will only be maximised if high-dose molecules can also be delivered in therapeutic doses using MNs. Suitably formulated dissolving MN platforms can deliver therapeutic doses of a low potency, high dose drug substance [16].

Thus, there remains a strong need for biocompatible, robust and effective drug-delivery microneedles, and improved approaches to the manufacture of such microneedles.

Microneedles containing a base and penetrating tip, and including active pharmaceutical ingredients, are described in US2017/0252546, WP2011/071287. CN105726458 and CN107349518. In all of the microneedles described in these documents, greater than 90% by weight of the microneedle constitutes non-pharmaceutical polymer that is required to provide the mechanical strength needed to penetrate the skin, meaning that less than 10% by weight of the microneedle is active pharmaceutical agent.

It is an object of the invention to overcome at least one of the above-referenced problems.

### SUMMARY OF THE INVENTION

The problems of low drug loading in microneedles has been addressed by identifying drugs (i.e. active pharmaceutical ingredients or API's) that are suitable for melting by heating to a molten form, and forming robust microneedles from the molten drugs in which the microneedle predominantly or almost completely consists of the drug. The Applicants have discovered that a heat-meltable API that is solid at 25° C., and has an ability to form a glassy, amorphous form following melting and cooling, where the amorphous form has a glass transition temperature greater than 25° C., are suitable for forming microneedles in the absence of any stabilising material. Microneedles formed from heat-meltable active pharmaceutical ingredient, in which more than 90%, and in particular 95% to 99%, of the microneedle comprises the meltable active pharmaceutical ingredient, are described herein.

In a first aspect, the invention provides a microneedle body comprising at least 80% meltable active pharmaceutical ingredient (v/v), typically capable of providing sustained release of the active pharmaceutical ingredient over a period of time after insertion of the microneedle or microneedles into the body (i.e. skin, nail or epithelium)

In one embodiment, the microneedle is substantially or completely free of non-pharmacologically active stabilising material. Examples of non-pharmacologically active stabilising material includes polymers (i.e. dissolvable polymers such as PLGA), carbohydrates (i.e. maltose), and resins.

In one embodiment, the microneedle is substantially or completely free of polymer, carbohydrate or both. Thus, the microneedle typically does not include the usual stabilising polymers, resins or carbohydrates conventionally employed in microneedles.

In one embodiment, the microneedle comprises at least 85%, 90%, 95%, 98% or 99% meltable active pharmaceutical ingredient (v/v).

In one embodiment, the meltable active pharmaceutical ingredient is selected from an antifungal, a corticosteroid, or a Non-steroidal class of anti-inflammatory agent. Examples of meltable drug suitable for use in the present invention are provided in Table 1 below:

TABLE 1

| | MELTING | GLASSY AND | GLASS |
|---|---|---|---|
| | TEMP | AMORPHOPUS | TRANSITION |
| DRUG | (° C.) | FORM | TEMP (TG ° C.) |
| Antifungal | | | |
| Clotrimazole | 148 | YES | 32 |
| Ketoconazole | 150 | YES | 46 |
| Itraconazole | 166 | YES | 49-63 |
| Fluconazole | 139 | YES | 32 |
| Corticosteroids | | | |
| Estradiol | 151 | YES | 85 |
| Betamethazone | 183 | YES | 54 |
| valerate | | | |
| Anti-inflamatory | | | |
| Sulindac | 182 | YES | 75 |
| Diclofenac | 157 | YES | 53 |
| Indomethacin | 160 | YES | 45 |
| Celecoxib | 158 | YES | 58 |

In another aspect, the invention provides a microneedle composition comprising a microneedle according to the invention and a microneedle backing layer containing a water soluble polymer attached to a base of the microneedle. Examples of biodegradable polymers include polylactic acid or a derivative thereof such as an ester terminated polylactide, polyglycolic acid or a derivative thereof such as an ester terminated polyglycolide, or polylactic co-glycolic acid or a derivative thereof such as an ester terminated polylactide co-glycolide. Examples of microneedle backing layers are described in WO2016/155891 (Leo Pharma).

In another aspect, the invention provides a microneedle drug delivery system (DDS), comprising:

a substrate; and

one or more microneedles according to the invention integrated or attached to, and extending from, the substrate,

wherein each microneedle typically comprises a base and a penetrating tip.

In one embodiment, the microneedle drug delivery system comprises a fast dissolving microneedle backing layer containing a water soluble polymer disposed between at least one of the microneedles and the substrate. Examples of microneedle backing layers are described in WO2016/155891 (Leo Pharma).

In any embodiment, the or each microneedle, independently, ranges from about 15 μm to about 1500 μm in length.

In any embodiment, the or each microneedle, independently, ranges from about 150 μm to about 1000 μm in length.

In any embodiment, the length of at least one of the microneedles is different from the others.

In another aspect, the invention provides a method of fabricating a microneedle or microneedle drug delivery system, comprising the steps of:

melting a meltable active pharmaceutical ingredient; and

shaping the molten meltable active pharmaceutical ingredient to form a microneedle.

The molten meltable active pharmaceutical ingredient may be shaped by moulding in a micromold, by 3-D printing techniques, or by low volume dispensing techniques, for example hot-melt low volume fluid dispensing technology.

In one embodiment, the method comprising the steps of:

providing a microneedle micromold comprising a micromold substrate and one or more holes in the upper surface of the micromold substrate, wherein the interior surface of the hole in the micromold substrate defines an exterior surface of the microneedle;

moulding a meltable drug in the microneedle micromold to form a microneedle; and separating the microneedle from the microneedle micromold.

In one embodiment, the meltable active pharmaceutical ingredient is added to the micromold in a solid, particulate, form, and melted in the micromold at a melting temperature.

In another embodiment, the meltable active pharmaceutical ingredient is melted and added to the micromold in a molten form.

In one embodiment, vacuum or centrifugation is employed to promote the meltable, melting or molten active pharmaceutical ingredient filling the micromold.

In one embodiment, the meltable active pharmaceutical ingredient in a solid particulate form is placed on a substrate, and the microneedle micromold is placed on the solid particulate meltable active pharmaceutical ingredient with the upper surface of the micromold abutting the active pharmaceutical ingredient, prior to melting and/or cooling of the meltable active pharmaceutical ingredient in the micromold under vacuum or centrifugation.

In one embodiment, the active pharmaceutical ingredient is melted and dispensed into the micromold or on to a substrate using low volume fluid dispensing technology. Examples of such fluid dispensers are commercially available from Poly-Pico Technologies Limited (PicoPRECISE). In one embodiment, the low volume fluid dispensing technology employs low volume high precision acoustic dispensing techniques. Low volume fluid dispensers are described in European Patent Application EP2613889.

In one embodiment, the active pharmaceutical ingredient is melted and dispensed using a hot melt dispenser, in which the active pharmaceutical ingredient is melted in the dispenser and then dispensed from the dispenser in a molten fluid form. Examples of such dispensers include the UNITY™ PURJET™ dispensing systems, the MAX II DISPENSE SYSTEM (GDP Global), and the HV-2000 Jet system from ADVANJET™.

In one embodiment, the molten active pharmaceutical ingredient is dispensed on to a substrate into a microneedle shape by 3-D printing. The use of 3-D printing for forming microneedles is described in the literature, for example in Pere et al. (International Journal of Pharmaceutics, Vol. 544, Issue 2, 425-432), Luzuriaga et al. (Lab Chip, 2018; 18(8)) and Farias et al. (Bioengineering 2018, 5, 59).

In another aspect, the invention provides a microneedle (or microneedle drug delivery system) formed according to a method of the invention, in which the microneedle is typically characterised by comprising at least 80% meltable active pharmaceutical ingredient, having an amorphous, glass form, and a glass transition temperature of greater than 25° C.

In another aspect, the invention provides a microneedle or microneedle composition or microneedle drug delivery system of the invention (or formed according to a method of the invention), for use in a method of treating a disease or condition for which the active pharmaceutical ingredient is indicated. The method comprises applying the microneedle (or microneedle composition or microneedle DDS) to the patient, for example a body surface of the patient such as the skin or nail surface or mucosal surface, whereby the API is administered to the patient. In one embodiment, the invention provides for the treatment of nail infections, especially fungal nail infections, in which the API is an anti-fungal agent.

In another aspect, the invention provides an electrospun fibre comprising at least 80%, 85%, 90% or 95% meltable active pharmaceutical ingredient (v/v). The electrospun fibre may be formed by hot melt electrospinning (Zhang et al. RSC Advances, Issue 58, 2016, and Long et al. Electrospinning: Nanofabrication and Applications, 2019).

Other aspects and preferred embodiments of the invention are defined and described in the other claims set out below.

### BRIEF DESCRIPTION OF THE FIGURES

FIGS. 1A and 1B illustrate microneedles according to the invention formed on a substrate.

FIG. 2 illustrates a method of fabricating a microneedle drug delivery device of the invention.

FIG. 3 illustrate Powder X-Ray Diffraction PXRD of a meltable drug (Itraconazole). PXRD employed to determine the formation of amorphous structure.

FIG. 4: The LC-MS analysis revealed that there is a high stability of the ITZ before and after melting for microneedle fabrication. The same fragmentation pattern was observed for the tested samples (ITZ, ITZ melt).

FIG. 5: Analysis of ¹H and ¹³C NMR confirmed the chemical structure of ITZ, with molecular formula C₃₅H₃₈Cl₂N₈O₄. No difference was observed for the 3 analyzed samples of ITZ, ITZ melt1 and ITZ melt2. Which indicating the chemical stability of both ITZ melts.

FIG. 6 illustrate the Differential Scanning calorimetry (DSC) of a meltable drug (Itraconazole). DSC employed to determine the glass transition temperature (Tg) of a meltable drug in amorphous form.

FIGS. 7A-D illustrate the Scanning Electron microscope SEM of the different microneedles according to the invention formed on a substrate: 5A—Itraconazole; 5B—Clotrimazole; 5C—Indomethacin; 5D—Estradiol.

FIG. 8: The force-distance curve for ITZ microneedle array measured by the Texture Analyzer.

FIGS. 9 A&B: Images of the pig ear skin treated with the microneedle array patch. The holes created by insertion of a hypodermic needle confirm breach of the skin surface at the site of application by the MN arrays, which will allow the therapeutics to be delivered.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

One aspect provided herein relates to microneedles formed from meltable active pharmaceutical ingredient. Such microneedles each have a base and a penetrating tip, wherein the penetrating tip has a dimension ranging from about 50 nm to about 50 μm.

As used therein, the term “penetrating tip” refers to an end of a microneedle that is adapted to first contact and penetrate a surface, e.g., of a biological barrier. The penetrating tip can be of any shape and/or dimension. The penetrating tip can have a shape of various geometries, e.g., but not limited to, circles, rectangles, squares, triangles, polygons, and irregular shapes. In some embodiments, the penetrating tip can appear as a point, for example, due to limited resolution of optical instruments, e.g., microscopes, and/or of human eyes. In some embodiments, the shape of the penetrating tip can be the same as or different from that of the cross section of the microneedle body.

The term “dimension” as used herein generally refers to a measurement of size in the plane of an object. With respect to a penetrating tip of the microneedles described herein, in some embodiments, the dimension of a penetrating tip can be indicated by the widest measurement of the shape of the penetrating tip. For example, the dimension of a circular tip can be indicated by the diameter of the circular tip. In accordance with the invention, the penetrating tip can have a dimension (e.g., a diameter) ranging from about 50 nm to about 50 μm, including from about 100 nm to about 40p m, from about 200 nm to about 40 μm, from about 300 nm to about 30 μm, from about 500 nm to about 10 μm, or from about 1 μm to about 10 μm. In some embodiments, the penetrating tip can have a dimension (e.g., a diameter) ranging from about 50 nm to about 10 μm, e.g., from about 50 nm to about 8 μm, from about 100 nm to about 5 μm, or from about 100 nm to about 2 μm. In other embodiments, the penetrating tip can have a dimension (e.g., a diameter) of less than 50 nm, or greater than 50 μm. Compared to previous polymer-based dissolvable microneedle designs (generally with a penetrating tip having a dimension of more than 10 μm [9]), some embodiments of the microneedles described herein can have sharper tips (e.g., less than 10 μm, 5 μm or 2 μm), thus increasing the probability of each microneedle penetrating a tissue (e.g., skin) and in turn increasing the overall amount of an active agent administered into the tissue.

The base of the microneedles described herein is generally the opposite end of the penetrating tip. The base of the microneedles can be attached or secured to a solid substrate or a device for facilitating the penetration of the microneedles into a biological barrier, optionally via a backing layer containing a water soluble polymer. The base of the microneedle can be of any size and/or shape. The base can have a shape of various geometries, e.g., but not limited to, circles, rectangles, squares, triangles, polygons, and irregular shapes. In various embodiments, the shape of the base can follow that of the cross section of the microneedle body.

Generally, the base of the microneedles described herein is the widest portion of the microneedles. However, in some embodiments, the base and the body of the microneedles can have substantially the same width. In some embodiments, the base, the body and the penetrating tip of the microneedle can have substantially the same width. A skilled artisan can determine an appropriate base dimension based on a number of factors, including, but not limited to, the length and aspect ratio of the microneedle body, the type of surfaces to be penetrated, and mechanical property of the drug. In some embodiments, the base dimension (e.g., a diameter) of the microneedles can range from 50 nm to about 1500 μm, from about 50 nm to about 1000 μm, from about 100 nm to about 750 μm, from about 250 nm to about 500 μm, or from about 500 nm to about 500 μm.

The microneedles described herein can be in any elongated shape suitable for use in tissue piercing, with minimal pain to a subject. For example, without limitations, the microneedle can be substantially cylindrical, wedge-shaped, cone-shaped, pyramid-shaped, irregular-shaped or any combinations thereof.

The shape and/or area of the cross section of the microneedles described herein can be uniform and/or vary along the length of the microneedle body. The cross-sectional shape of the microneedles can take a variety of shapes, including, but not limited to, rectangular, square, oval, circular, diamond, triangular, elliptical, polygonal, U-shaped, or star-shaped. In some embodiments, the cross section of the microneedles can have a uniform shape and area along the length of the microneedle body. In some embodiments where the microneedles are irregular-shaped, their cross sections can vary in both shape and area along the length of the microneedle body, or their cross sections can vary in shape (with a constant area) along the length of the microneedle body. In one embodiment, the microneedles described herein comprise a tapered body with a substantially circular cross section along the length of the microneedle body. The cross-sectional dimensions of the microneedle body can range from 0.05 μm to about 1500 μm, from about 0.05 μm to about 1000 μm, from about 0.1 μm to about 750 μm, from about 0.25 μm to about 500 μm, or from about 0.5 μm to about 500 μm.

The length of the microneedle body can vary from micrometers to centimeters, depending on a number of factors, e.g., but not limited to, types of tissue targeted for administration, required penetration depths, lengths of the uninserted portion of a microneedle, and methods of applying microneedles across or into a biological barrier. By way of example only, if a microneedle is required to reach into a few centimeters of an organ tissue (e.g., heart tissue) during surgery, the microneedle can be of several centimeters long. In such embodiments, the microneedle can be further secured to an applicator or a device for facilitating the penetration of the microneedle into the organ tissue (e.g., heart tissue). Thus, some embodiments of the microneedles described herein can have a length of about 0.5 cm to about 10 cm, about 1 cm to about 8 cm, or about 2 cm to about 6 cm.

In some embodiments, the length of microneedle body can vary from about 10 μm to about 5000 μm, from about 50 μm to about 2500 μm, from about 100 μm to about 1500 μm, from about 150 μm to about 1000 μm, or from about 200 μm to about 800 μm. In some embodiments, the length of microneedle body can vary from about 200 μm to about 800 μm. By way of example, some embodiments of the microneedles described herein can be used for skin penetration. The skin's outermost barrier, the stratum corneum, is generally about 10 μm to 20 μm thick, and covers the viable epidermis, which is about 50 μm to 100 μm thick. The epidermis is avascular, but it hosts Langerhan's cells (immature myeloid dendritic cells) which can be, for example, relevant in inducing an immune response, e.g., immunization. Below these skin layers, the dermis is about 1 mm to 2 mm thick and houses a rich capillary bed, which can be a useful target for systemic delivery of an active agent. The robust mechanical properties of meltable drug allow construction of microneedles that penetrate the skin to any appropriate depth. For example, the length of microneedles can be constructed long enough to deliver an active agent to the viable epidermis (about 10 μm to 120 μm below the skin surface), e.g., to induce an immune response. In some embodiments, the length of microneedles can be constructed long enough to deliver an active agent to the dermis (about 60 μm to 2.1 mm below the skin surface). An ordinary artisan can adjust the microneedle length for a number of factors, including, without limitations, tissue thickness, e.g., skin thickness, (as a function of age, gender, location on body, species (animals), drug delivery profile (e.g., fast—long needle vs. slow—short needle), diffusion properties of active agents (e.g., ionic charge, molecule weight, shape), or any combinations thereof. A microneedle length can range between about 50 μm to about 700 μm, depending on the tissue targeted for administration. In some embodiments, devices with individual microneedles ranging in sizes from 15 μm to 300 μm can be fabricated.

Accordingly, the length of the microneedle body can be selected and constructed for each particular application. In some embodiments, the length of the microneedle body can further comprise an uninserted portion, i.e. a portion of the microneedle that is not generally involved in tissue penetration. In those embodiments, the length of the microneedle body can comprise an insertion length (a portion of a microneedle that can penetrate into or across a biological barrier) and an uninserted length. The uninserted length can depend on applications and/or particular device designs and configurations (e.g., a microneedle adaptor or a syringe that holds a microneedle).

The microneedle is generally substantially or completely free of non-pharmacologically active stabilising material. Examples of non-pharmacologically active stabilising material includes polymers (i.e. dissolvable polymers such as PLGA), carbohydrates (i.e. maltose), and resins. “Microneedle drug delivery system” or “microneedle DDS” means a substrate bearing one or more microneedles according to the invention integrated or attached to, and extending from, the substrate, wherein each microneedle typically comprises a base and a penetrating tip.

The term “drug” is art-recognized and refers to any chemical moiety that is a pharmacologically active substance that acts locally or systemically in a subject. It is also known as an “active pharmaceutical ingredient” or “API”. The terms “drug” and “active pharmaceutical ingredient” or “API” are used interchangably herein. Examples of drugs are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject.

As used herein, the term “meltable drug” or “meltable active pharmaceutical ingredient” refers to an active pharmaceutical ingredient that can be heat-melted to a molten form and has the following properties:
solid at 25° C.;
ability to form a glassy, amorphous form following heat-melting and cooling; and
the amorphous form having a glass transition temperature typically greater than 25° C.

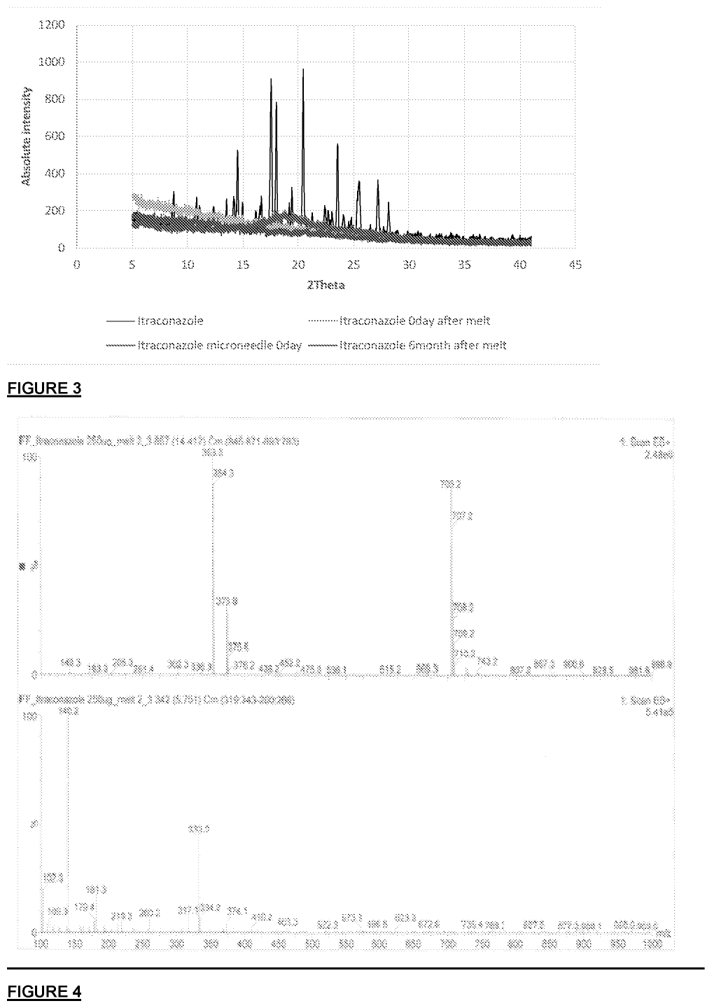

Examples of meltable drugs include; antifungals (for example, Itraconazole, Clotrimazole, Ketoconazole, Fluconazole, or derivatives or variants thereof), corticosteroids (for example, Estradiol, betamethasone valerate or derivatives or variants thereof), Anti-inflammatory drugs (for example, Celecoxib, Diclofenac, Sulindac, Indomethacin), Antimicrobials and Antibiotics (for example, Cefuroxime exetil, Chloramphenicol or derivatives or variants thereof), Cardiovascular and Antihypertensives (Carvedilol, Nifedipine or derivatives or variants thereof), Autonomic nervous system and Psychiatrics medications (Droperidol or derivatives or variants thereof), Antilipidemic and Cholesterol medications (Probucol, Simvastatin or derivatives or variants thereof), Gastrointestinal medications (Famotidine, Omeprazole or derivatives or variants thereof), Respiratory medications, Endocrine medications, Immunomodulators, Oncology drugs, Renal medications, Neurologic medications and anti-migraine (Zolmitriptan or derivatives or variants thereof). Typically, the meltable drug forms a stable amorphous form following melting and cooling; in this context, the formation of amorphous structure is confirmed by the XRD technique. X-ray powder Diffraction (XRD) is an analytical technique primarily used for phase identification of a crystalline material, in which the crystalline structure causes a beam of incident X-rays to diffract into many specific directions. It works best for materials that are crystalline or partially crystalline (i.e., that have periodic structural order) but is also used to study non-crystalline materials FIG. 3.

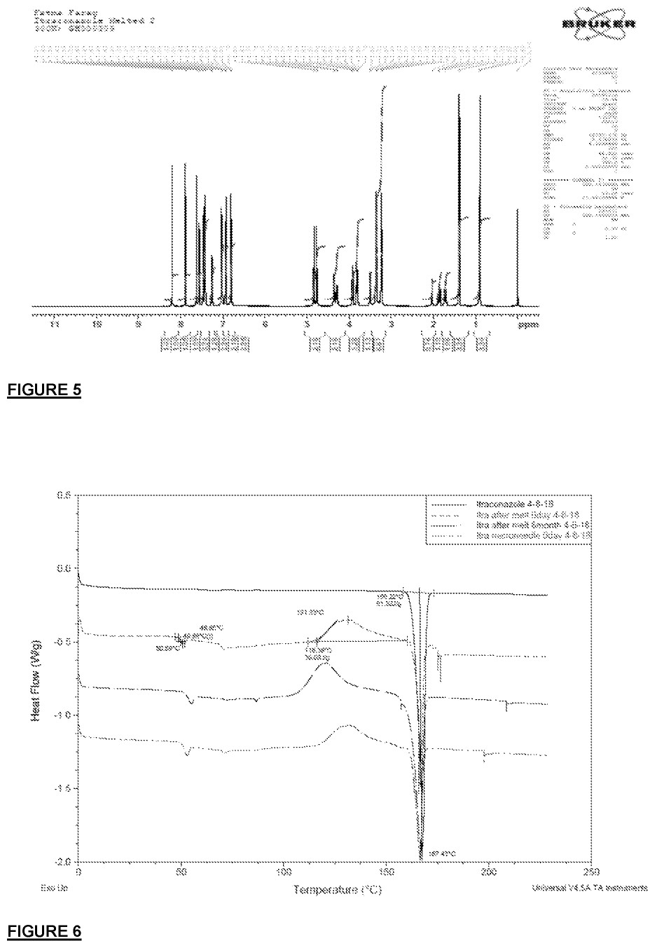

The meltable active pharmaceutical ingredient forms a stable amorphous form following melting and cooling; in this context, the term “stable” as applied to the amorphous form means that the thermodynamic tendency of the active pharmaceutical ingredient to crystallize over one year is resisted. Typically, the meltable active pharmaceutical ingredient exhibits minimal degradation during melting and cooling. In this context, minimal degradation means that at least 90% of the active pharmaceutical ingredient retains its therapeutic activity following melting and cooling. The LC-MS analysis revealed that there is a high stability of the ITZ before and after melting for microneedle fabrication (FIG. 4). The same fragmentation pattern was observed for the tested samples (ITZ, ITZ melt). Also, analysis of ¹H and ¹³C NMR confirmed the chemical structure of ITZ, with molecular formula C₃₅H₃₈Cl₂N₈O₄. No difference was observed for the 3 analyzed samples of ITZ, ITZ melt1 and ITZ melt2. Which indicating the chemical stability of both ITZ melts (FIG. 5). Differential Scanning calorimetry (DSC) may be employed to determine the glass transition temperature (Tg) of a meltable drug in amorphous form DSC utilizes a heat flow technique and compares the amount of heat supplied to the test sample and a similarly heated “reference” to determine transition points. Tg is typically calculated by using a half-height technique in the transition region FIG. 6.

The term “antifungal agent” as used herein refers to a substance capable of inhibiting or preventing the growth, viability and/or reproduction of a fungal cell. In some embodiments, antifungal agents include those capable of preventing or treating a fungal infection in an animal or plant. An antifungal agent can be a broad-spectrum antifungal agent or an antifungal agent specific to one or more particular species of fungus. Non-limiting examples of antifungal agents include ergosterol synthesis inhibitors such as azoles (e.g., imidazoles and triazoles) and phenpropimorph, and terbinafine. The term “azole” as used herein refers to a class of 5-membered heterocyclic compounds containing a nitrogen atom and at least one other non-carbon atom (i.e. nitrogen, sulfur, or oxygen). Examples of azoles include ketoconazole, itraconazole, fluconazole, clotrimazole, voriconazole, posaconazole, ravuconazole and miconazole. Typically, the meltable drug is a synthetic azole.

As used herein, the term “amorphous” as applied to the meltable active pharmaceutical ingredient in the microneedle of the invention means that it is a non-crystalline solid in which the atoms and molecules are not organized in a definite lattice pattern.

As used herein, the term “glassy” as applied to the meltable active pharmaceutical ingredient in the microneedle of the invention means that it is an amorphous solid that exhibits a glassy behaviour, i.e. mechanically rigid, at temperatures below its temperature. The glass transition temperature is the gradual and reversible transition in amorphous materials from a hard “glassy” state into a rubbery-like state as the temperature is increased.

Another aspect provided herein is a microneedle drug delivery system (DDS) comprising a substrate and one or more microneedles described herein integrated or attached to the substrate and extending from the substrate, wherein each microneedle comprises a base and a penetrating tip. In some embodiments, the microneedle DDS can comprise a substrate and a microneedle. In some embodiments, the microneedle DDS can comprise a substrate and at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100 or more microneedles.

Each microneedle present on the microneedle DDS need not to have the same microneedle length. In some embodiments, each microneedle on the microneedle DDS can have the same microneedle body length. In alternative embodiments, the microneedles on the microneedle DDS can have different microneedle body lengths. Thus, a predefined profile of constant or varying microneedle depth penetrations can be provided in a single microneedle DDS. In some embodiments, the body length of each microneedle can be tuned to adjust for the curvature of a surface.

A plurality of microneedles can be arranged in a random, pseudo-random or predefined pattern, such as an array. The distance between the microneedles and the arrangement of the plurality of microneedles can be selected according to the desired mode of treatment and characteristics of the treatment site. For example, in some embodiments, a sub-population of microneedles can be arranged closely together as a group, e.g., to increase the amount of active agent delivered to a target spot.

The microneedles can be oriented perpendicular or at an angle to the substrate. In some embodiments, the microneedles can be oriented perpendicular to the substrate. In such embodiments, a larger density of microneedles per unit area of substrate can be provided. Substrate: The substrate of the microneedle DDS can be constructed from a variety of materials, including metals, ceramics, semiconductors, organics, polymers, and any composites thereof. The substrate includes the base substrate to which the microneedles are attached or integrally formed. The substrate can then be adapted to fit a Luer-Lock syringe or other conventionally used drug delivery device that currently uses hypodermic needles as the barrier penetration method.

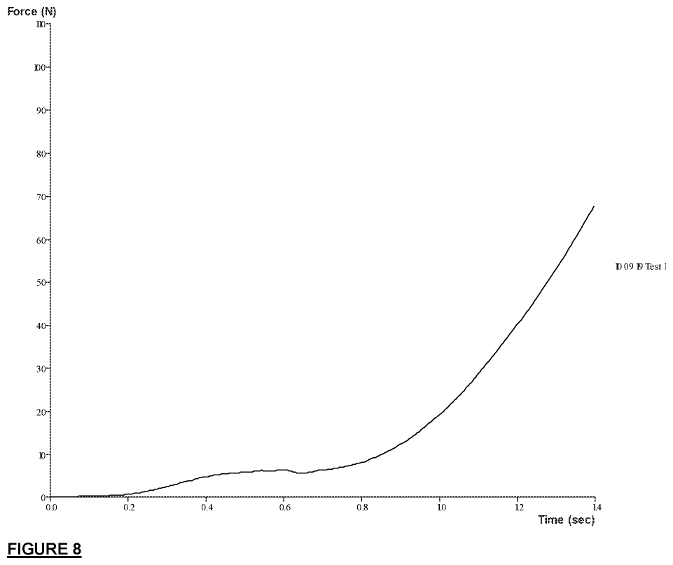

To prevent the microneedles from breaking on insertion into the skin, the mechanical strength of the microneedles should be such that the force required to fracture the microneedle is significantly greater than the force required to insert the microneedle into the skin. Generally, the force required to insert a microneedle patch into the skin and have it penetrate past the stratum corneum is in the range of 0.4-8N, for instance 2-7N, such as 5N, per patch containing 25 microneedles per cm. The failure force of the microneedle can be assessed as either a fracture force or the force required to compress the microneedle by a defined length. These forces can be can be determined using a texture analyser (e.g. a TA.XT Plus Texture Analyzer, Stable Micro Systems, Surrey, UK). Texture Analyzer was used to apply forces using a metal probe to base-plates placed between two aluminium blocks. A maximum peak observed in the force-distance curve represented the force required to break the base plate. As it is possible to see from the graph (FIG. 8), the force used to stress the microneedles path is around 8 N and it is means that they are strong.

In some embodiments of the device, the substrate can comprise one or more biocompatible polymers. By the term “biocompatible polymer” meant is a polymeric material which when in contact with a human body does not provoke an adverse response in the subject. Examples of biocompatible polymers include, but are not limited to, silicone and silicone-based polymers; polytetrafluoroethylene (PTFE); a natural or synthetic hydrogel; polyurethane; polysulfone; cellulose; polyethylene; polypropylene; polyamide; polyester; polymethylmethacrylate, polylactic acid (PLA), polyglycolic acid (PGA), poly(lactic-co-glycolic acid) (PLGA), polyvinyl alcohol (PVA), polyvinyl pyrollidone (PVP), carboxymethyl cellulose (CMC), ethylcellulose (EC), methyl cellulose (MC) any art-recognized biocompatible polymers, and any combinations thereof.

In some embodiments of the microneedle DDS, the substrate can comprise one or more biodegradable polymers, e.g., but not limited to, poly(lactide)s, poly(glycolide)s, poly(lactide-co-glycolide)s, polyanhydrides, polyorthoesters, polyetheresters, polycarpolactones, polyesteramides, poly(butyric acid)s, poly(valeric acid)s, polyhydroxyalkanoates, degradable polyurethanes, cellulose derivatives any copolymers thereof, and any blends thereof.

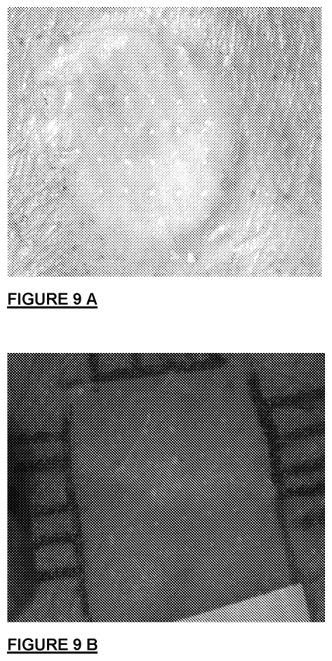

In some embodiments of the microneedle DDS, the substrate can be formed from any flexible material. In such embodiments, the substrate can be sufficiently flexible to conform to a surface upon contact with the surface, e.g., a tissue or an organ surface, while allowing the microneedles to penetrate the tissue to the desired depth (FIG. 9). In one embodiment, the flexible substrate comprises a film integrated with microneedles. In alternative embodiments, the substrate can be any rigid material.

The surface of the substrate from which the microneedles extend can be a substantially flat surface, a curved surface, a wavy surface or any combinations thereof. In some embodiments, the surface of the substrate from which the microneedles extend can be configured to have a curvature profile similar to that of a target surface to be penetrated. The substrate can be of any shape and/or any dimension determined from, for example, design of the microneedle DDS, area/shape of a target site to be treated, and/or size of microneedle applicators. In some embodiments, the shape and dimension of the substrate can be configured to fit any applicator that currently uses hypodermic needles as the barrier penetration method (e.g., syringes), any microinjection equipment, any microneedle holders, any microneedle administration or applicator devices, any microneedle array applicator devices, and/or microneedle array cartridge systems. Non-limiting examples of the microneedle or microneedle array injectors or applicators include the ones described in U.S. Patent Application Nos.: US 2008/0183144; US 2003/0208167; US 2010/0256597; and U.S. Pat. Nos. 6,743,211; and 7,842,008.

The microneedles may be made by melting the meltable active pharmaceutical ingredient and then forming microneedles from the molten active pharmaceutical ingredient. The microneedles may be formed by dispensing the molten active pharmaceutical ingredient into a micromold and cooling to form the microneedles, which are then released from the micromold. Vacuum or centrifugation may be employed to ensure that the molten drugs fills the micromold. The meltable active pharmaceutical ingredient may be melted and dispensed using a hot melt dispenser. The microneedles may also be formed by 3-D printing of the molten drug. The microneedles may be formed by hot melt electrospinning of a drug fibre, which can be shaped into a microneedle shape, for example by spooling on to a microneedle shaped mandrel. In one embodiment, the invention provides a 3-D printed microneedle comprising at least 80% meltable drug (v/v). In one embodiment, the invention provides an electrospun fibre comprising at least 80% meltable drug (v/v).

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1—Formation of Microneedles from Meltable Anti-Fundal (Itraconazole)

A melt method in a vacuum oven was used to fabricate Itraconazole microneedle DDS. The oven used was a Memmert oven connected to a Pfeiffer D-35614 Asslar vacuum. The oven temperature was set to 5° C. above the melting point of the examined medication. The melting approach was applied by placing the API, itraconazole, on a glass slide, and then, placing the microneedles substrate (moulds), with the holes facing downwards on the top of the API powder. Place the slide into the vacuum oven, and set the temperature to 171° C. (5° C. above the melting temperature of the API) and the pressure to 10 mbar (using the temperature and vacuum functions) to allow the drug to melt. Once the temperature has reached the desired temperature, and the drug is visibly melting, leave it for 10-15 minutes and then release the pressure. Once the microneedles are cool, use a blade to gently remove any excess drug from the surface of the mould.

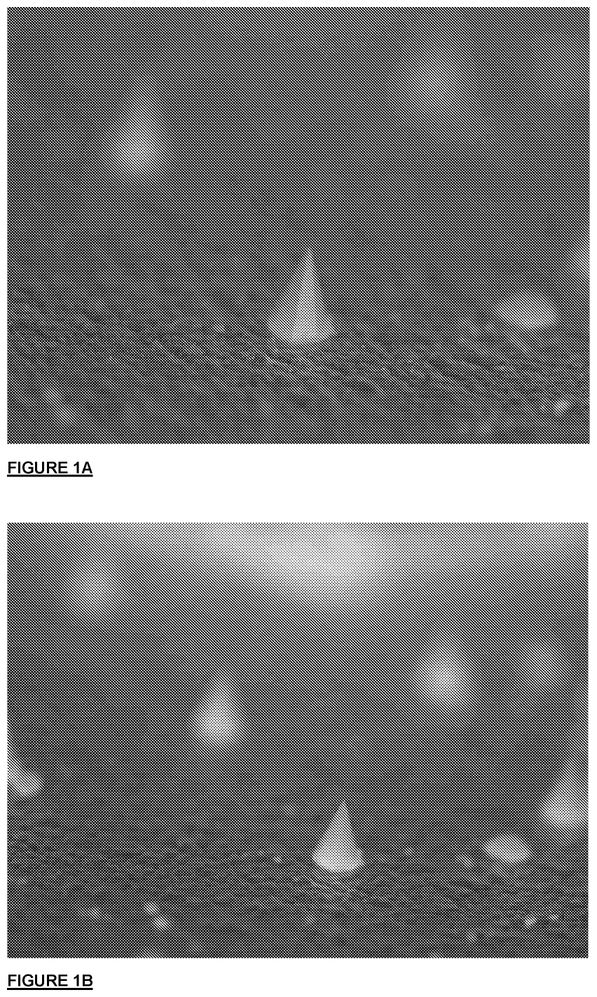

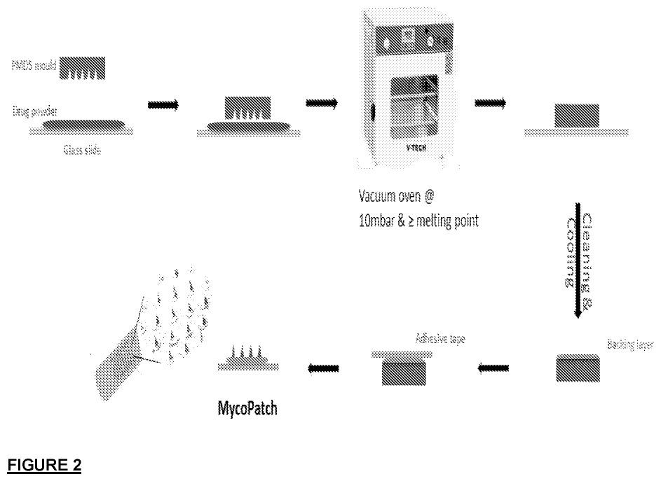

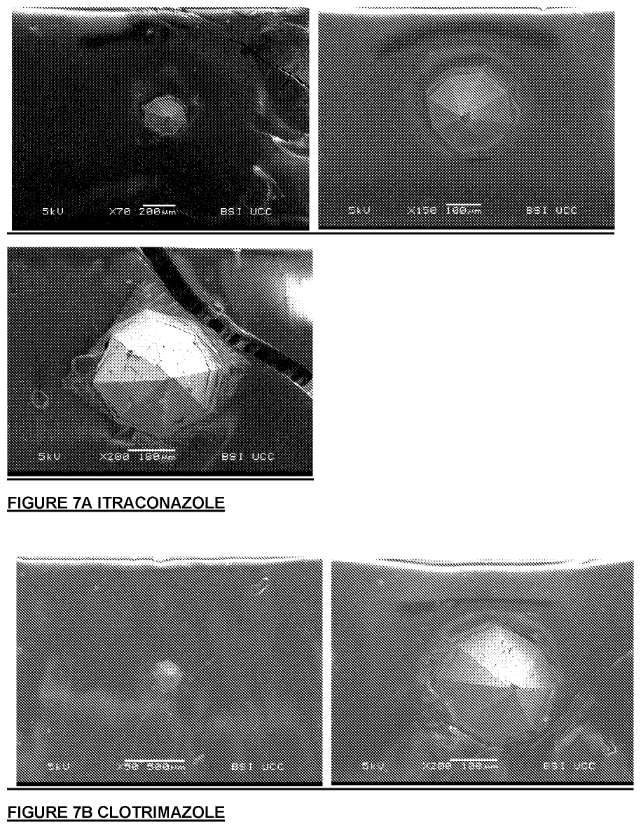

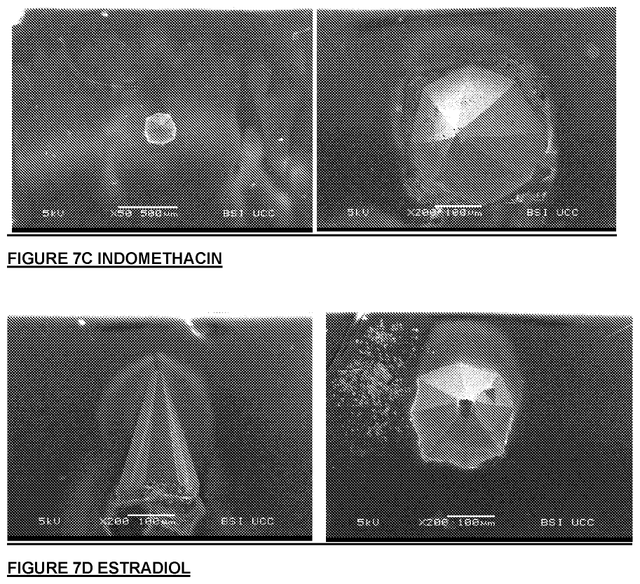

The microneedles may then be removed from the mould by applying adhesive tape on top of the mould and applying pressure to ensure good contact between the tape and the base of the microneedles followed by pulling the microneedles out of the mould. The tape should preferably be adhesive medical tape as this has been found to provide good adhesion to the base of the microneedles so that substantially all microneedles are removed from the mould when the tape is pulled. The resulting microneedles were visually characterised using an Olympus Optical light microscope with imaging view 7software, FIG. 1, and Scanning Electron microscope SEM, FIG. 7A.

### Example 2—Formation of Microneedles from Meltable Non-Steroidal Anti-Inflammatory (Indomethacin)

A melt method in a vacuum oven was used to fabricate Indomethacin microneedle DDS. The oven used was a Memmert oven connected to a Pfeiffer D-35614 Asslar vacuum. The oven temperature was set to 5° C. above the melting point of the drug of interest, indomethacin. The melting approach was applied by placing the API, Indomethacin, on a glass slide, and then, placing the microneedles substrate (moulds), with the holes facing downwards on the top of the API powder. Place the slide into the vacuum oven, and set the temperature to 165° C. (5° C. above the melting temperature of the API) and the pressure to 10 mbar (using the temperature and vacuum functions) to allow the drug to melt. Once the temperature has reached the desired temperature, and the drug is visibly melting, leave it for 10-15 minutes and then release the pressure. Once the microneedles are cool, use a blade to gently remove any excess drug from the surface of the mould.

The microneedles may then be removed from the mould by applying adhesive tape on top of the mould and applying pressure to ensure good contact between the tape and the base of the microneedles followed by pulling the microneedles out of the mould. The tape should preferably be adhesive medical tape as this has been found to provide good adhesion to the base of the microneedles so that substantially all microneedles are removed from the mould when the tape is pulled. The resulting microneedles were visually characterised using an Olympus Optical light microscope with imaging view 7software, and Scanning Electron microscope SEM, FIG. 7C.

### Example 3—Formation of Microneedles from Meltable Corticosteroid (Estradiol)

A melt method in a vacuum oven was used to fabricate Estradiol microneedle DDS. The oven used was a Memmert oven connected to a Pfeiffer D-35614 Asslar vacuum. The oven temperature was set to 5° C. above the melting point of the drug of interest, Estradiol. The melting approach was applied by placing Estradiol on a glass slide, and then, placing the microneedles substrate (moulds), with the holes facing downwards on the top of the API powder. Place the slide into the vacuum oven, and set the temperature to 156° C. (5° C. above the melting temperature of the API) and the pressure to 10 mbar (using the temperature and vacuum functions) to allow the drug to melt. Once the temperature has reached the desired temperature, and the drug is visibly melting, leave it for 10-15 minutes and then release the pressure. Once the microneedles are cool, use a blade to gently remove any excess drug from the surface of the mould.

The microneedles may then be removed from the mould by applying adhesive tape on top of the mould and applying pressure to ensure good contact between the tape and the base of the microneedles followed by pulling the microneedles out of the mould. The tape should preferably be adhesive medical tape as this has been found to provide good adhesion to the base of the microneedles so that substantially all microneedles are removed from the mould when the tape is pulled. The resulting microneedles were visually characterised using an Olympus Optical light microscope with imaging view 7software, and Scanning Electron microscope SEM, FIG. 7d.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCES

1. Gill, H. S. and M. R. Prausnitz, Pocketed microneedles for drug delivery to the skin. Journal of Physics and Chemistry of Solids, 2008. 69(5-6): p. 1537-1541.
2. Li, W. Z., et al., Super-short solid silicon microneedles for transdermal drug delivery applications. Int J Pharm, 2010. 389(1-2): p. 122-9.
3. Sullivan, S. P., N. Murthy, and M. R. Prausnitz, Minimally invasive protein delivery with rapidly dissolving polymer microneedles. Advanced Materials, 2008. 20(5): p. 933-+.
4. Poon, H. Y., et al., Zeolite microneedles for controlled transdermal drug delivery. Abstracts of Papers of the American Chemical Society, 2013. 246.
5. Martanto, W., et al., Transdermal delivery of insulin using microneedles in vivo. Pharmaceutical Research, 2004. 21(6): p. 947-952.
6. Chen, Y., et al., Fabrication of coated polymer microneedles for transdermal drug delivery. Journal of Controlled Release, 2017. 265: p. 14-21.
7. Gill, H. S. and M. R. Prausnitz, Coated microneedles for transdermal delivery. Journal of Controlled Release, 2007. 117(2): p. 227-237.
8. Kim, Y. C., J. H. Park, and M. R. Prausnitz, Microneedles for drug and vaccine delivery. Advanced Drug Delivery Reviews, 2012. 64(14): p. 1547-1568.
9. Park, J. H., M. G. Allen, and M. R. Prausnitz, Polymer microneedles for controlled-release drug delivery. Pharmaceutical Research, 2006. 23(5): p. 1008-1019.
10. Ito, Y., et al., Evaluation of self-dissolving needles containing low molecular weight heparin (LMWH) in rats. International Journal of Pharmaceutics, 2008. 349(1-2): p. 124-129.
11. Li, G. H., et al., In vitro transdermal delivery of therapeutic antibodies using maltose microneedles. International Journal of Pharmaceutics, 2009. 368(1-2): p. 109-115.
12. Miyano, T., et al., Hydrolytic microneedles as transdermal drug delivery system. Transducers '07 & Eurosensors Xxi, Digest of Technical Papers, Vols 1 and 2, 2007.
13. Lee, J. W., J. H. Park, and M. R. Prausnitz, Dissolving microneedles for transdermal drug delivery. Biomaterials, 2008. 29(13): p. 2113-2124.
14. Donnelly, R. F., et al., Hydrogel-Forming Microneedle Arrays for Enhanced Transdermal Drug Delivery. Adv Funct Mater, 2012. 22(23): p. 4879-4890.
15. McCrudden, M. T., et al., Strategies for enhanced peptide and protein delivery. Ther Deliv, 2013. 4(5): p. 593-614.
16. McCrudden, M. T. C., et al., Design and physicochemical characterisation of novel dissolving polymeric microneedle arrays for transdermal delivery of high dose, low molecular weight drugs. Journal of Controlled Release, 2014. 180: p. 71-80.

1-21. (canceled)

22. A method of fabricating a microneedle, comprising the steps of:
melting active pharmaceutical ingredient,
shaping the molten active pharmaceutical ingredient into a microneedle shape; and
cooling the molten active pharmaceutical ingredient to form a microneedle,
wherein the active pharmaceutical ingredient is meltable, solid at 25° C., and has an ability to form a glassy, amorphous form following melting by heating and cooling with a glass transition temperature greater than 25° C.

23. The method according to claim 22, in which the active pharmaceutical ingredient constitutes at least 80% of the microneedle (v/v).

24. The method according to claim 22, in which the microneedle consists of active pharmaceutical ingredient.

25. The method according to claim 22, comprising the steps of:
providing a microneedle micromould comprising a micromould substrate and one or more holes in the upper surface of the micromould substrate, wherein the interior surface of the hole in the micromould substrate defines an exterior surface of the microneedle;
moulding the active pharmaceutical ingredient in the microneedle micromould to form the microneedle; and
separating the microneedle from the microneedle micromould,

26. The method according to claim 22, comprising the steps of:
providing a microneedle micromould comprising a micromould substrate and one or more holes in the upper surface of the micromould substrate, wherein the interior surface of the hole in the micromould substrate defines an exterior surface of the microneedle;
moulding the active pharmaceutical ingredient in the microneedle micromould to form the microneedle; and
separating the microneedle from the microneedle micromould,
in which the meltable active pharmaceutical ingredient is added to the micromold in a solid, particulate form, and melted to a molten form in the micromould at a melting temperature.

27. The method according to claim 22, comprising the steps of:
providing a microneedle micromould comprising a micromould substrate and one or more holes in the upper surface of the micromould substrate, wherein the interior surface of the hole in the micromould substrate defines an exterior surface of the microneedle;
moulding the active pharmaceutical ingredient in the microneedle micromould to form the microneedle; and
separating the microneedle from the microneedle micromould,
the method including the steps of placing the active pharmaceutical ingredient in a solid particulate form on a substrate, placing the microneedle micromould on the solid particulate active pharmaceutical ingredient with the upper surface of the micromould abutting the active pharmaceutical ingredient, and melting by heating and then cooling the active pharmaceutical ingredient in the micromold.

28. The method according to claim 22, comprising the steps of:
providing a microneedle micromould comprising a micromould substrate and one or more holes in the upper surface of the micromould substrate, wherein the interior surface of the hole in the micromould substrate defines an exterior surface of the microneedle;
moulding the active pharmaceutical ingredient in the microneedle micromould to form the microneedle; and
separating the microneedle from the microneedle micromould,
in which the active pharmaceutical ingredient is melted and added to the micromould in a molten form.

29. The method according to claim 22, comprising the steps of:
providing a microneedle micromould comprising a micromould substrate and one or more holes in the upper surface of the micromould substrate, wherein the interior surface of the hole in the micromould substrate defines an exterior surface of the microneedle;
moulding the active pharmaceutical ingredient in the microneedle micromould to form the microneedle; and
separating the microneedle from the microneedle micromould,
in which the method includes a step of applying vacuum or centrifugal forces to the micromould to promote the active pharmaceutical ingredient filling the micromold.

30. The method according to claim 22, in which the active pharmaceutical ingredient is an antifungal drug selected from Itraconazole, Clotrimazole, Ketoconazole, and Fluconazole.

31. The method according to claim 22, in which the active pharmaceutical ingredient is a steroid selected from estradiol, and betamethasone valerate.

32. The method according to claim 22, in which the active pharmaceutical ingredient is an anti-inflammatory drug selected from Celecoxib, Diclofenac, Sulindac, and Indomethacin.

33. The method according to claim 22, in which the active pharmaceutical ingredient is an antimicrobial and antibiotic drug selected from Cefuroxime exetil and Chloramphenicol.

34. The method according to claim 22, in which the active pharmaceutical ingredient is a cardiovascular and antihypertensive drug selected from Carvedilol and Nifedipine.

35. The method according to claim 22, in which the active pharmaceutical ingredient is an autonomic nervous system and Psychiatric drug, optionally Droperidol.

36. The method according to claim 22, in which the active pharmaceutical ingredient is an antilipidemic and cholesterol lowering drug selected from Probucol and Simvastatin.

37. The method according to claim 22, in which the active pharmaceutical ingredient is a gastrointestinal drug selected from Famotidine and Omeprazole.

38. The method according to claim 22, in which the active pharmaceutical ingredient is a neurologic or anti-migraine drug, optionally Zolmitriptan.

39. The method according to claim 22, in which the active pharmaceutical ingredient comprises a plurality of active pharmaceutical ingredients.
